(19) United States
(12) Patent Application Publication
(10) Pub. No.: US 2002/0103110 A1
(43) Pub. Date: Aug. 1, 2002

(54) System and method for rectal administration of medication for treatment of migraines

(76) Inventor: A. Robert Spitzer, Southfield, MI (US)

Correspondence Address: KOHN & ASSOCIATES, Suite 410, 30500 Northwestern Highway, Farmington Hills, MI, 48334, US

(21) Appl. No.: 10/056,734

(22) Filed: Jan. 25, 2002

Related U.S. Application Data
(60) Provisional application No. 60/264,413, filed on Jan. 26, 2001.
(60) Provisional application No. 60/302,799, filed on Jul. 3, 2001.

(51) Int. Cl. A61K 31/00; A61M 5/178

(52) U.S. Cl. 514/1; 604/217; 604/167.02

(57) ABSTRACT

The present invention provides for a system for treating migraine headaches including an administering mechanism for administering a medication composition, a rectal adapting mechanism operatively connected to the administering mechanism for insertion through an anus and into a rectum of a patient, and a medication composition contained within the administering mechanism. The present invention also provides for a medicine suppository including a medication composition having an effective amount of valproate for the treatment of a migraine headache. Furthermore, the present invention provides for a kit including the system of the present invention. Finally, the present invention provides for a method of treatment of a migraine by rectal administration of an effective amount of a medication composition.

### CROSSREFERENCE TO RELATED APPLICATIONS

This application claims the benefit of priority under 35 U.S.C. Section 119(e) of U.S. Provisional Patent Application No. 60\264,413, filed Jan. 26, 2001, and U.S. Provisional Patent Application 60\302,799, filed Jul. 3, 2001, which are incorporated herein by reference in their entirety.

### TECHNICAL FIELD

The present invention relates to the field of medical devices and medical treatment. More particularly, the present invention relates to a system and method for treatment of migraine headaches.

### BACKGROUND ART OF THE INVENTION

A migraine is a severely painful syndrome characterized by recurring headache resulting from cerebral vasoconstriction. Millions of people suffer from migraine headaches. The common migraine usually begins as a slowly developing pain that transforms into a mounting and throbbing pain. Typically, the pain is accompanied by nausea and sometimes vomiting. There are numerous causes for a migraine including, but not limited to, stress, anxiety, anger, worry, overexertion, food-related reactions, and other sensory-related reactions.

There are numerous types of therapeutic drugs that are currently used to alleviate the pain of a migraine. These anti-migraine drugs include, but are not limited to, ergot alkaloids, beta-blocking agents, ergotamine or ergotamine-like agents, serotonin agonists, 5HT agonists, calcium channel blocking agents, antidepressants, local anesthetics, adrenergic blocking agents, sedatives, caffeine with ergots, non-prescription analgesics such as acetaminophen, aspirin, ibuprofen, non-steroidal anti-inflammatory agents such as naproxen and naproxen sodium, and mixtures thereof. Various patents disclose other effective medication compositions. For instance, U.S. Pat. No. 6,159,505 to Piper discloses compositions for the treatment of migraine or stress headaches wherein there is supplied a combination of potassium, magnesium, and pyridoxine optionally in association with other nutrients and/or simple analgesics. U.S. Pat. No. 5,753,712 to Pinsker discloses a treatment for migraine headaches utilizing a compound or a pharmaceutically acceptable acid addition salt thereof. Another formulation described in U.S. Pat. No. 4,380,540 to Poyser et al. discloses aspirin intermixed with metoclopramide.

The above mentioned medications or agents can be introduced through various routes including the oral route and parenteral route by intravenous injection. Oral treatment usually is not effective until two to three hours after administration and does not provide the fastest method of achieving pain relief. Intravenous injection is faster, but is more inconvenient and invasive. Unfortunately, these two routes present additional problems and side effects in conjunction with the drugs themselves. Some significant side effects include chest pain, nausea, vomiting, body pains, drowsiness, dizziness, diarrhea, vertigo, and possible numbness.

Another route however, is by rectal administration. Rectal administration provides for an alternative, yet effective route of administration. Due to the thin walls of the rectum and abundance of blood vessels located therein, the rectum provides for a fast and effective route of administration. Moreover, the rectal administration does not produce the side effects associated with other parenteral and oral routes.

Thus, there needs to be a more effective device that administers effective amounts of medication to treat migraines wherein a reduced amount of side effects occur, while still producing rapid and effective treatment. Additionally, there needs to be a method associated therewith to effectively treat the same.

### SUMMARY OF THE INVENTION

According to the present invention, there is provided a system for treating migraine headaches including an administering mechanism for administering a medication composition, a rectal adapting mechanism operatively connected to the administering mechanism for insertion through an anus and into a rectum of a patient, and a medication composition contained within the administering mechanism. The present invention also provides for a medicine suppository including a medication composition having an effective amount of valproate for the treatment of a migraine headache. Furthermore, the present invention provides for a kit including the system of the present invention. Finally, the present invention provides for a method of treatment of a migraine by rectal administration of an effective amount of a medication composition.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 a side-view of an embodiment of the system of the present invention;

FIG. 2 is an enlarged side-view of an embodiment of the rectal adapting mechanism of the present invention;

FIG. 3 is a side view of another embodiment of the system of the present invention, wherein a cartridge mechanism containing a pre-measured dosage of medication composition is utilized;

FIG. 4 is a side-view of the cartridge mechanism of the present invention;

FIG. 5 is a side-view of another embodiment of the present invention being in a form of a suppository system; and

FIG. 6 is a side-view of another embodiment of the present invention, wherein the administering mechanism is a bulb.

### DETAILED DESCRIPTION OF THE INVENTION

Generally, the present invention provides for a system 10 and method for the treatment of migraine headaches. While specific embodiments are disclosed herein, they are not exhaustive and can include other suitable designs that utilize other similar administrative devices used in combination with other rectal adaptings for insertion into the rectum of a patient. In other words, the present invention can utilize the administrative devices and associated adapters disclosed herein, or the present invention can utilize any administrative devices and associated adapters already known to those of skill in the art. Additionally, various types of medications can be utilized with the system disclosed herein. Basically, any differing design, structure, and composite materials known to those of skill in the art can be utilized without departing from the spirit of the present invention.

The present invention can be used in any number of settings that include, but are not limited to, medical offices, hospitals, emergency rooms, nursing homes, schools, private homes and any other similar environments. Additionally, use of the present invention can occur with or without the aid of any medical personnel or other individual. Moreover, the present invention is useful for inhibiting or moderating a migraine headache, while also being able to be used to prevent the onset of a migraine headache attack.

The critical aspect of the present invention is the route of administration. Rectal administration of migraine treating medications or medication compositions 16 provide for fast and effective relief of the symptoms and pain associated with a migraine headache. Rectal administration has numerous advantages. For instance, due to the abundance of blood vessels located within and surrounding the rectum, medication can effectively and quickly enter the bloodstream. Moreover, the walls of the rectum are thin and substances such as medication can easily pass and rapidly absorb into the bloodstream therein. Additionally, the various side effects, such as nausea, that are associated with other administration routes can be avoided by use of rectal administration.

The term “medication composition” 16 as used herein is meant to include a pharmaceutical composition that includes a medication. The medication includes, but is not limited to, valproate, sodium valproate, valproate salts, relproic acid, ergotamine, ergotamine-like agents, serotonin agonists, 5HT agonists, caffeine with ergots, aspirin, acetaminophen, naproxen sodium, tolfenamic acid, ibuprofen, other pharmacological agents, and any other similar prescription and nonprescription drugs known by those of skill in the art that can treat migraines or other similar disorders. As is described herein, a wide variety of migraine medications or agents are known in the art. Thus, any migraine medication or agent can be utilized with the present invention. The medication composition 16 utilized herein can be in any form including, but not limited to, a liquid, gel, paste, viscous solution, suspension, solid, and any other similar form known to those of skill in the art. Preferably, the medication is a viscous solution or suspension, which is suitable for rectal insertion utilizing the present invention. Additionally, it would be useful for the medication composition 16 to be in a form that not only can be easily insertable into the rectum of a patient, but also is capable of being retained inside the patient's rectum without substantial leakage or drainage therefrom. The medication composition 16 can be made through various methods and standard chemical reactions known to those of skill in the art. The medication composition 16 of the present invention can be administered or applied to the rectum of a patient in anticipation of a potential or imminent migraine attack or during the migraine attack. While a single dose can be administered that is effective to inhibit the migraine, multiple doses can be periodically given.

As used herein, the term “therapeutically effective amount” of the medication refers to an amount of that medication that is physiologically significant and improves an individual's condition. A medication is “physiologically significant” if its presence results in a change in the physiology of the recipient individual. For example, in the treatment of a migraine headache, administration of a medication that relieves or arrests further progress of the condition would be considered both physiologically significant and therapeutically effective.

Specifically referring to the medication composition 16, it can include various substances in addition to the medication itself. Other substances include, but are not limited to, thickeners, suitable pharmaceutically-acceptable carriers, solvents, physiologically-acceptable preservatives, and any other suitable substances known to those of skill in the art. The medication composition optionally can include an amount of a thickener for rendering the consistency of the composition effective for rectal administration to a patient. Preferably, the medication has a viscosity such that it can be quickly administered by injection, yet once administered, and does not tend to leak out of or drain from the anus. As a result, the medication composition remains and spread on the surface of the rectal mucosa for a period of time that is sufficient for absorption into the bloodstream of the patient. Examples of thickeners include, but are not limited to, cellulose ethers such as methylcellulose, carboxy-methylcellulose, hydroxypropylcell ulose, hydroxypropylmethylcellulose, biogums, carboxy vinyl polymers, and any other similar thickeners known to those of skill in the art. The amount of a thickener selected can be determined by routine experimentation. Typically, the thickener constitutes less than 25% of the weight of the medication composition 16.

The medication composition 16 can also include a solvent that is used as a carrier of the medication. The solvent can be any suitable, pharmaceutically acceptable solvent known to those of skill in the art including, but not limited to, water, deionized water, distilled water, organic solvents, and mixtures thereof. As for the organic solvents, they can be included in the medication composition if certain water insoluble medications are utilized. Such organic solvents include, but are not limited to, nontoxic polyols, alkanols, propylene glycol, ethyl alcohol, and the like. Additional solvents include Hank's solution, Ringer's solution, physiologically buffered saline, sodium carboxymethyl cellulose, sorbitol, dextran, oils such as sesame oil, synthetic fatty acid esters such as ethyl oleate, triglycerides, or liposomes, non-lipid polycationic amino polymers, and the like. Additionally, stabilizers or agents can be added to increase the solubility of the compounds and allow for the preparation of highly concentrated solutions.

Generally, the solvents are used in amounts effective to solubilize the medication and to inhibit precipitation thereof. Typically, about 25 to 75 wt-% of a solvent can be employed. In some cases, the solvent used in the medication composition can consist entirely of one or more nontoxic organic solvents. Again, the amount of solvent used can be determined by routine experimentation.

As for the preservatives, they include, but are not limited to, benzyl alcohol, thimerosal, chlorobutanol, methyl parabens, propyl parabens, benzalkonium chloride, and any other similar preservatives known to those of skill in the art. The concentration of the preservative needed in a composition varies with the preservative selected. Usually, the preservative amount is determined by routine experimentation. A typical amount of preservative though, is usually less than 5% of the weight of the medication composition 16.

As for the medication, the dosage of the medication varies. As with any compound, the effective dose can be initially estimated either in cell culture assays (e.g., of neoplastic cells) or in animal models such as mice, rats, rabbits, dogs, or pigs. An animal model can also be used to determine the appropriate concentration range.

A therapeutically effective dose refers to that amount of active ingredient, which ameliorates the symptoms or conditions of a migraine headache. Therapeutic efficacy and toxicity can be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of therapeutic to toxic effects is the therapeutic index, and it can be expressed as the ED₅₀/LD₅₀ ratio. Pharmaceutical compositions that exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage can be determined by the practitioner, in light of factors related to the subject requiring treatment. Dosage and administration are adjusted to provide sufficient levels of the active moiety or to maintain the desired effect. Factors which can be taken into account include the severity of the condition, the general health of the subject, the age, weight, and gender of the subject, time and frequency of administration, drug combination(s), reaction sensitivities, and response to therapy. Long-acting pharmaceutical compositions can be administered every 3 to 4 days, every week, or biweekly depending on the half-life and clearance rate of the particular formulation.

Normal dosage amounts can vary from about 0.1 mg to 100,000 mg. Typical pre-loaded doses would be 750 mg, 1000 mg, and 1500 mg. Guidance as to particular dosages and methods of delivery is provided in the literature and generally available to practitioners in the art.

All ingredients contained in the medication composition are in concentrations determined as described above. Additionally, standard and routine procedures and reference to standard pharmacological texts and publications provide additional guidance. The medication composition can be prepared by mixing the ingredients according to generally accepted procedures for formulating pharmaceutical mixtures. Generally, the ingredients can be mixed in a mixer, blender, or other standard mixing device to produce a concentrated mixture. Sufficient time for mixing and procedures for elimination of lumps is used to ensure that a homogenous mixture is formed. Additional amounts of water can be added to obtain the desired concentration. The concentration of the buffering ingredients can also be adjusted to obtain the desired pH in the final composition. Alternatively, if the proper amounts of the various ingredients have been determined by prior experiments, these amounts can be added and mixed together in a mixing vessel without pH adjustment.

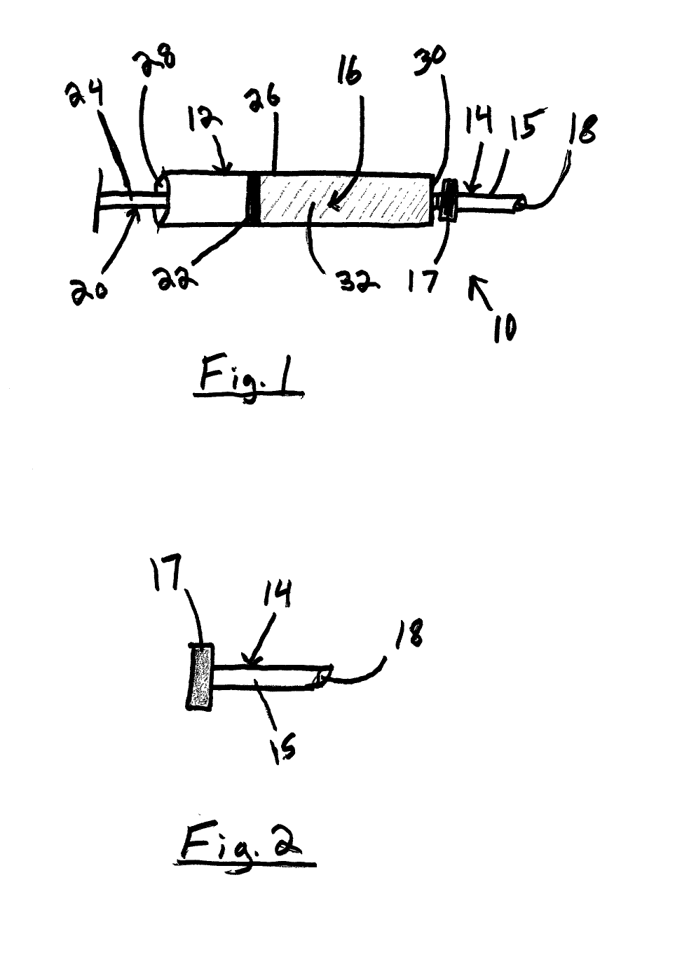

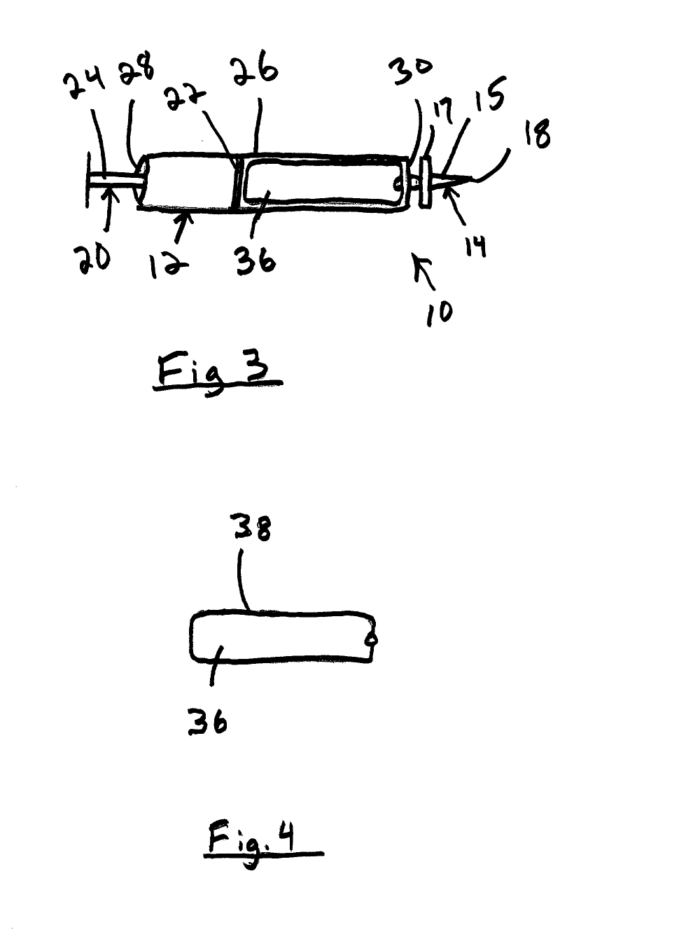

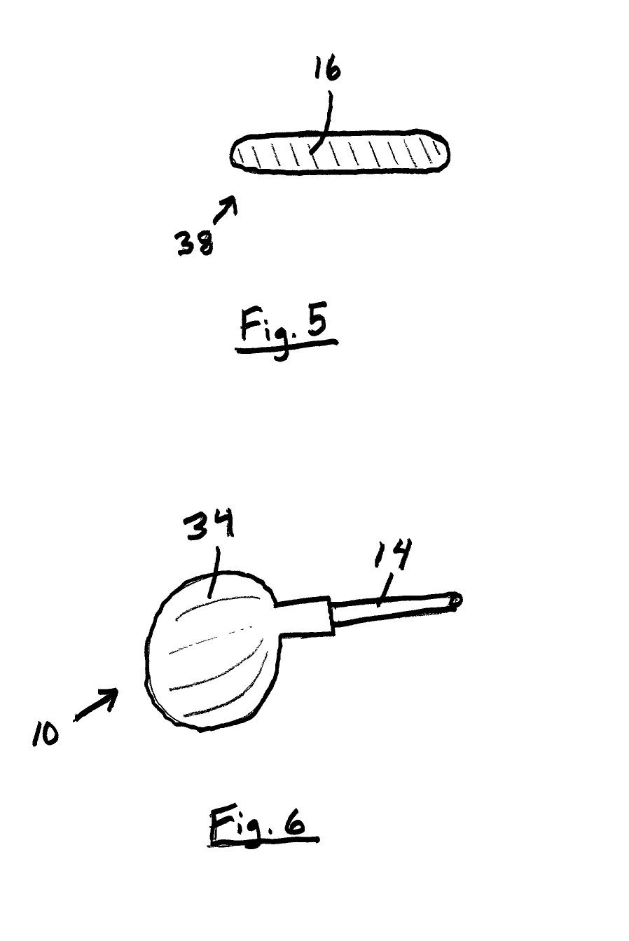

Referring now to the embodiments of the present invention, one embodiment provides for a system, generally indicated by 10, in FIG. 1. The system 10 includes an administering mechanism 12 for administering a medication composition, a rectal adapting mechanism 14 operatively connected to the administering mechanism 12 for insertion through an anus and into a rectum of a patient, and a medication composition 16 contained within the administering mechanism 14.

The administering mechanism 12 of the present invention is defined as, but is not limited to, a syringe, medical bulb, dispensing chamber, and any other similar syringe-type device known to those of skill in the art. The administering mechanism 12 can be any device that can hold desired amounts of medication composition 16 and subsequently dispense the medication composition 16 in a controlled manner. Various materials and combinations thereof can be used to construct the administering mechanism 12 that include, but are not limited to, plastic, metal, rubber, glass, and any other similar material known to those of skill in the art.

Preferably, the administering mechanism 12 is a syringe-like device having a barrel 26 wherein the barrel 26 has a first end 28 and second end 30. The first end 28 is open for insertion of the medication composition 16 therein and for insertion of a pressure mechanism 20. The second end 30 is operatively connected or engaged to the rectal adapting mechanism 14. This connection must be snug to prevent leakage of the medication composition 16 from the administering mechanism 12 during administration of the medication composition 16 thereof. The pressure mechanism 20 is associated with or operatively connected to the barrel 26 for forcing the medication composition 16 out of the barrel 26, through the rectal adapting mechanism 14, and into the rectum. Generally, the pressure mechanism 20 has a plunger end 22 and plunger rod 24 attached to the plunger end 22 at one end. The pressure mechanism 20 can slide on the surface of the lumenal wall of the barrel 26 of the administering mechanism 12. In one embodiment, the pressure mechanism 20 has a relatively soft deformable plunger head 22. The pressure mechanism 20 along with the barrel 26, and the second end of the administering mechanism 12 form a chamber 32 therein. More specifically, the chamber 32 is formed from the plunger end 22 of the pressure mechanism 20, the wall of the barrel 26 and the second end 30 of the barrel 26. The chamber 32 contains therein the medication composition 16.

Although the administering mechanism 12 is described as a syringe-like device, other embodiments are provided herein. For instance, the administering mechanism 12 can be a simple, flexible bulb-shaped body as shown in FIG. 6. The medication composition 16 can be located within the bulb 34 or other chamber operatively connected and in communication with the bulb 34. Dispensing the medication composition 16 can then be achieved by applying pressure to the exterior body of the bulb 34.

The rectal adapting mechanism 14 is defined as, but is not limited to, a repository needle, plastic tubing, rubber hosing, and any other similar elongated, hollow chamber known to those of skill in the art that can be inserted into the rectum of the patient. Materials and combinations thereof can be used to construct the rectal adapting mechanism 14 that include, but are not limited to, plastic, metal, rubber, and any other similar material known to those of skill in the art. The rectal adapting mechanism 14 is operatively connected and engaged to the administering mechanism 12 at the second end 30. The rectal adapting mechanism 14 can be simply a straight, but flexible tube, or can be tapered. The rectal adapting mechanism 14 can be any length, but is typically about 4 cm to about 9 cm long and has a blunt, open tip or outlet 18. Preferably, the tip 18 is blunt enough to obviate the risk of puncturing a patient's tissue when the rectal adapting mechanism 14 is inserted into the rectum, yet is small enough to facilitate easy insertion therein. It is also preferable that the rectal adapting mechanism 14 be tapered for easy insertion into the rectum. Such a rectal adapting mechanism 14 would facilitate the administration of the medication composition 16 into the rectum without causing injury to the individual.

The rectal adapting mechanism 14 includes an elongated infusion tube 15 having a tip or outlet 18 at the end opposite to the juncture with the administering mechanism 12. The infusion tube 15 is of varying length and is relatively stiff enough for insertion into the rectum. As stated above, the infusion tube 15 is preferably tapered from the juncture between the rectal adapting mechanism 14 and the second end 30 of the administering mechanism 12. The rectal adapting mechanism 14 further can include a collar 17 situated at the end proximate to the juncture with the administering mechanism 12. Basically, the collar 17 is a flange that extends radially outward from the infusion tube 15.

Another embodiment of the administering mechanism 12 of the present invention includes a cartridge mechanism 36. The cartridge mechanism 36 contains a pre-measured amount of medication composition 16 and is inserted into the chamber 32. Once the cartridge mechanism 36 is loaded into the chamber 32 therein, the pressure mechanism 20 is inserted into the first end 28 of the barrel 26 of the administering mechanism 12 so that the pressure mechanism 20 can effectively force the medication composition 16 out of the cartridge mechanism 36 and through the second end 30 of the administering mechanism 12. As a result, the medication composition 16 exits through the second end 30, into the rectal adapting mechanism 14, through the tip or outlet 18 located therein, and subsequently into the rectum of the patient.

Typically, the cartridge mechanism 36 has a hollow cylindrical body portion 38. The outside diameter of the cartridge is formed to a size that easily slips into the barrel bore of the barrel 26. The size of the cartridge mechanism 36 varies according to the amount of medication composition 16 desired and the size of the administering mechanism 12. The cartridge mechanism 36 can be made of many materials including, but not limited to, glass, metal, foil, plastic, and any other similar materials known to those of skill in the art. In operation, the cartridge mechanism 36 is activated by the pressure mechanism 20, and more specifically the plunger end 22. Once the pressure mechanism 20 is depressed by the user, the plunger end 22 engages the cartridge mechanism 36, which then dispenses the medication composition 16.

Yet another embodiment of the present invention is a suppository 38 that includes a medication composition 16 having an effective amount of a medication for effectively treating a migraine attack. As previously described, various medications can be utilized to effectively treat a migraine with the suppository 38 described herein. The main medication used with the suppository 38 is valproate and other valproate derivatives and/or salts thereof. Whatever the medication, the effective dosage of the medication composition 16 is enough to provide the patient with relief of the symptoms associated with a migraine attack. The suppository 38 can be in different forms including, but not limited to, a solid cone, cylinder, pill, and any other similar solid design that is known to those of skill in the art. Moreover, the suppository 38 should be readily and easily meltable within the patient. The suppository 38 is inserted into a bodily passage or cavity such as the rectum of an individual. The suppository 38 further includes other ingredients including, but not limited to, various carriers and substances including, but not limited to, glycerin and sodium stearate.

The suppository 38 is made by various methods known to those of skill in the art and can be prepared in such a manner. Further, the suppository 38 can be pre-packaged as a single or multi-dose unit for use by a patient. In operation, the suppository 38 is used by opening the package and inserting the suppository 38 into the rectum of the patient through the anus thereof.

Another embodiment of the present invention is a kit including the system 10 described herein that is preloaded with a pre-measured dose of the medication composition 16. The system 10 is preferably contained in suitable packaging, such as a box, envelope or plastic blister pack, along with suitable instructions for its use. The loaded one-dose application functions as a single unit dosage form of the medication composition that can be administered by the patient or by another. The prepackaged kit has an advantage of providing fast administration of the medication composition. A person only needs to open the package of the kit, remove the system 10 from the package, and follow enclosed instructions. Depending on the type of kit, the system 10 can already contain the loaded medication composition 16 and the person only need to break any seal that is present on the system 10 itself, insert the tip 18 of the rectal adapting mechanism 14 in the rectum of the patient, and apply pressure to the pressure mechanism 20 of the administering mechanism 12 to deliver the medication composition 16 into the rectum of the patient. Alternatively, the kit can include a separate pre-measured cartridge mechanism 36 of medication composition 16 that can be loaded into the administering mechanism 12. The administering mechanism 12 is then used to deliver the medication composition 16 from the cartridge mechanism 36 to the rectum. In this case, the administering mechanism 12 can be reusable and the cartridge mechanism 36 can be disposable. The volume of the medication composition 16 prepackaged in the administering mechanism 12 can vary according to categories of body weight, body size, age, and any other appropriate factors known to those of skill in the art.

In operation, the present invention further provides a method of treating migraines using system of the present invention. Generally, the method of treating a migraine headache includes the step of administering an effective amount of a medication composition into the rectum of an individual. Alternatively, the method includes the steps of loading an effective amount of a medication composition 16 within the system 10 described herein and administering the medication composition 16 into the rectum of a patient. More specifically, the administering step is defined as inserting the rectal adapting mechanism 14 into the anus of the patient and depressing the pressure mechanism 20 of the administering mechanism 10.

Throughout this application, various publications, including United States patents, are referenced by author and year. All patents are referenced by their issued patent number. Full citations for the publications are listed below. The disclosures of these publications and patents in their entireties are hereby incorporated by reference into this application in order to describe more fully the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is therefore, to be understood that within the scope of the described invention, the invention may be practiced otherwise than as specifically described.

What is claimed is:

1. A system for treating migraine headaches comprising:
administering means for administering a medication composition;
rectal adapting means operatively connected to said administering means for insertion through an anus and into a rectum of a patient; and
a medication composition including an effective amount of a medication that can effectively treat migraine headaches of the patient, said medication composition being contained within said administering means.

2. The system according to claim 1, wherein said administering means is selected from the group consisting of a syringe, a medical bulb, and a depressible chamber.

3. The system according to claim 2, wherein said administering means is made from material selected from the group consisting of plastic, metal, rubber, glass, and combinations thereof.

4. The system according to claim 2, wherein said syringe is defined as a body having a cylindrical barrel with a first end and a second end, wherein said first end is open for insertion of said medication composition and said second end is engaged to said rectal adapting mechanism.

5. The system according to claim 4, wherein said syringe further includes pressure means operatively inserted into said first end for providing pressure for subsequent dispersion of said medication composition.

6. The system according to claim 5, wherein said pressure means is defined as a plunger having a plunger seal and plunger rod.

7. The system according to claim 6, wherein said pressure means, said cylindrical barrel, and said second end form a chamber for containing said medication composition therein.

8. The system according to claim 1, wherein said administering means includes cartridge means for containing a pre-measured dosage of medication composition, said cartridge is inserted into said cylindrical barrel.

9. The system according to claim 8, wherein said cartridge means is made from material selected from the group consisting of plastic, metal, glass, and foil.

10. The system according to claim 1, wherein said rectal adapting means is selected from the group consisting of a repository needle, plastic tubing, rubber hosing, and elongated hollow chamber.

11. The system according to claim 1, wherein said rectal adapting means is defined as an elongated tube having a distal end and an outlet in the vicinity of the distal end, for introducing the medication composition through the anus and into the rectum through said outlet.

12. The system according to claim 11, wherein said outlet end is blunt.

13. The system according to claim 11, wherein said rectal adapting means is made from material selected from the group consisting of plastic, metal, rubber, and combinations thereof.

14. The system according to claim 1, wherein said medication composition includes effective amounts of medicine selected from the group consisting of valproate, sodium valproate, valproate salts, relproic acid, ergotamine, ergotamine-like agents, serotonin agonists, 5HT agonists, caffeine with ergots, aspirin, acetaminophen, naproxen sodium, tolfenamic acid, and ibuprofen.

15. The system according to claim 14, wherein said medication is an effective amount of appropriate medication for the effective treatment of a migraine headache.

16. The system according to claim 15, wherein said effective amount is selected from the group consisting of 750 mg, 1000 mg, and 1500 mg.

17. The system according to claim 16, wherein said medication composition is in a form selected from the group consisting of a liquid, gel, paste, viscous solution, suspension, and solid.

18. The system according to claim 17, wherein said medication composition includes additional ingredients selected from the group consisting of thickeners, solvents, preservatives, and combinations thereof.

19. The system according to claim 18, wherein said thickeners are selected from the group consisting of cellulose ethers, methylcellulose, carboxymethylcellulose, hydroxypropylcellulose, hydroxypropylmethylcellulose, biogums, and carboxy vinyl polymers.

20. The system according to claim 18, wherein said solvents are selected from the group consisting of water, deionized water, distilled water, organic solvents, saline, and oils.

21. The system according to claim 18, wherein said preservatives are selected from the group consisting of benzyl alcohol, thimerosal, chlorobutanol, methyl parabens, propyl parabens, and benzalkonium chloride.

22. A medicine suppository comprising a medication composition including an effective amount of valproate for the treatment of a migraine headache.

23. The medicine suppository according to claim 22, wherein said valproate is selected from the group consisting of valproate, sodium valproate, valproate salts, and valproate variations thereof.

24. The medicine suppository according to claim 22, wherein said medication composition further includes medicine selected from the group consisting of relproic acid, ergotamine, ergotamine-like agents, serotonin agonists, 5HT agonists, caffeine with ergots, aspirin, acetaminophen, naproxen sodium, tolfenamic acid, and ibuprofen.

25. The medicine suppository according to claim 23, wherein said medication is an effective amount of appropriate medication for the effective treatment of a migraine headache.

26. The medicine suppository according to claim 25, wherein said effective amount is selected from the group consisting of 750 mg, 1000 mg, and 1500 mg.

27. The system according to claim 22, wherein said medication composition includes additional ingredients selected from the group consisting of thickeners, solvents, and preservatives.

28. A kit for treatment of a migraine headache comprising a system including administering means for administering a medication composition, rectal adapting means operatively connected to said administering means for insertion through an anus and into a rectum of a patient, and a medication composition including an effective amount of a medication that can effectively treat migraine headaches of the patient, said medication composition being contained within said administering means.

29. The kit according to claim 28, wherein said administering means includes cartridge means for containing a pre-loaded and pre-measured effective amount of the medication composition.

30. A method of treating a migraine headache by administering an effective amount of a medication composition into the rectum of an individual.

31. A method of treatment of a migraine by:
loading an effective amount of a medication composition within an apparatus having an administration mechanism operatively connected to a rectal adapting mechanism, the administration mechanism further including a pressure mechanism; and
administering the medication composition into the rectum of a patient.

32. The method according to claim 31, wherein said administering step is defined as inserting the rectal adapting mechanism into the anus of a patient and depressing the pressure mechanism of the administration mechanism.
